(19) United States
(12) Patent Application Publication
(10) Pub. No.: US 2016/0002347 A1
(43) Pub. Date: Jan. 7, 2016

(54) PYRUVATE KINASE M2 NEUTRALIZING ANTIBODIES FOR INHIBITING ANGIOGENESIS

(71) Applicant: PRODA BIOTECH LLC, Marietta, GA (US)

(72) Inventors: Zhi-Ren Liu, Marietta, GA (US); Liangwie Li, Atlanta, GA (US); Yinwei Zhang, Atlanta, GA (US)

(21) Appl. No.: 14/379,124

(22) PCT Filed: Feb. 14, 2013
(86) PCT No.: PCT/US2013/026146

§ 371 Date: Aug. 15, 2014

Related U.S. Application Data
(60) Provisional application No. 61/599,226, filed on Feb. 15, 2012.

(51) Int. Cl. C07K 16/40

(52) U.S. Cl. CPC C07K 16/40; C07K 2317/76; A61K 2039/505

(57) ABSTRACT

Methods for inhibiting angiogenesis, such as tumor angiogenesis, in a subject are disclosed. Pharmaceutical compositions for use in the disclosed methods are also described.

### PRIOR RELATED APPLICATION DATA

This application claims priority to U.S. Provisional Patent Application Ser. No. 61/599,226, filed Feb. 15, 2012, which is incorporated by reference in its entirety.

### STATEMENT REGARDING FEDERALLY SPONSORED RESEARCH OR DEVELOPMENT

This invention was made with Government Support under Grant No. CA118113 awarded by the National Institutes of Health. The Government may have certain rights in the invention.

### BACKGROUND

This disclosure is generally related to the field of neutralizing antibodies, more particularly to compositions and methods for inhibiting angiogenesis by neutralizing circulating pyruvate kinases M2.

### BACKGROUND OF THE INVENTION

Cancer drugs designed to starve tumors of their blood supply are called “angiogenesis inhibitors.” One class of these anti-angiogenesis drugs works by blocking the action of an essential protein known as vascular endothelial growth factor (VEGF), which normally stimulates new blood vessel growth. These drugs succeed at first, but then promote more invasive cancer growth, sometimes with a higher incidence of metastases. If the tumor cannot build its vasculature to a sufficient level, it should not spread and become invasive.

### SUMMARY

Compositions and methods for inhibiting angiogenesis in a subject are disclosed. These methods are based on the discovery that 1) soluble pyruvate kinase isoform M2 (PKM2) promotes tumor angiogenesis and 2) neutralizing circulating PKM2 effectively inhibits cancer growth. The disclosed methods involve administering to the subject a composition containing an effective amount of soluble PKM2 binding molecules in a pharmaceutically acceptable excipent.

In one specific embodiment, the PKM2 inhibitor binding molecules specifically binds and neutralizes circulating PKM2 in the subject. Therefore, a suitable subject for treatment has detectable levels of PKM2 in a bodily fluid or stool when the PKM2 inhibitor binding molecules is administered. In one specific embodiment, the PKM2 binding molecule is an antibody that binds or specifically binds and neutralizes PKM2, such as human PKM2. Antibodies can be whole immunoglobulin or immunoglobulin fragments containing at least the antigen binding region. Antibodies can be isolated from animal or human subjects, produced by gene recombination, or synthesized using routine methods. In other specific embodiments, the antibody is a human, human chimeric, or humanized antibody. Other molecules that bind proteins and that can function like antibodies can be used in the disclosed methods. In some embodiments, the PKM2 binding molecule is a peptide that binds and neutralizes PKM2.

The disclosed methods can be used to inhibit angiogenesis in any subject in need thereof. Angiogenesis is required for the growth and metastasis of cancer. Therefore, in some embodiments, the subject has cancer and the method is used to inhibit angiogenesis in the cancer. Angiogenesis in the eye underlies the major causes of blindness in both developed and developing nations. In some embodiments, the subject has exudative age-related macular degeneration (AMD) and the method inhibits angiogenesis in the eye of the subject.

Pharmaceutical compositions for use in the disclosed methods are also described. In some embodiments, the composition contains neutralizing antibodies that specifically bind and neutralize PKM2 in a pharmaceutically acceptable carrier. The PKM2 binding molecule is present in the composition in an effective amount to bind to PKM2 in the blood of a human.

### BRIEF DESCRIPTION OF THE DRAWINGS

FIG. 1A is a graph showing tumor volume (mm³) of SW620 tumor in mice treated with purified IgGs of PKM2 (PabPKM2) (-▪-) or pre-immune serum (PabCon) (-▴-) as a function of time (days) after inoculation. Tumor volumes were calculated by formula: tumor volume=π/6×(width)2×length.

FIG. 1B is a plot showing tumor weight (mg) of SW620 tumor in mice treated with PabPKM2 (left column) or PabCon (right column) after 13 days growth with 8 days treatment (treatment started 5 days post tumor inoculation).

FIG. 1C is a bar graph showing Ki-67 staining (percentage Ki67+) of tissue sections prepared from the harvested SW620 tumors treated with PabPKM2 (left column) or PabCon (right column).

FIG. 2A is a graph showing tumor volume (mm³) of SW620 tumor in mice treated with saline (-♦-), rPKM1 (-▪-), rPKM2 (-X-), or rPKM2+FBP (-▴-) as a function of time (days) after inoculation.

FIG. 2B is a plot showing tumor weight (mg) of SW620 tumor in mice treated with saline (column 1), rPKM1 (column 2), rPKM2 (column 3), or rPKM2+FBP (column 4).

FIGS. 3A and 3B are bar graph showing the number (average from four randomly selected fields from three slides) of branch points in endothelial tubes formed by HUVEC cells 1) in the presence of saline (FIG. 3A, column 1) rPKM1 (FIG. 3A, column 2), rPKM2 (FIG. 3A, column 3), or rPKM2+FBP (FIG. 3A, column 4), or 2) in the presence of culture medium collected from SW620 cells (620CM) (FIG. 3B, column 1), control culture medium (conCM) with addition of PabPKM2 (FIG. 3B, column 2), 620CM with addition of PabPKM2 (FIG. 3B, column 3) or 620CM with addition of PabCon (FIG. 3B, column 4).

FIGS. 4A and 4B are bar graphs showing cell proliferation (FIG. 4A) and migration (FIG. 4B) relative to saline control of HUVEC cells in the presence of buffer saline control (column 1), rPKM1 (column 2), rPKM2 (column 3), or rPKM2+FBP (column 4) analyzed by a commercial BrdU proliferation kit (FIG. 4A) and boyden chamber assay (FIG. 4B).

FIG. 4C is a bar graph showing cell attachment (relative to rPKM2) of HUVEC cells to cell culture plate on which BSA (column 1), rPKM1 (column 2), or rPKM2 (column 3) was coated. FIG. 4D is a bar graph showing cell attachment (relative to buffer saline) of HUVEC cells to cell culture plate on which fibronectin (open bars) or vitronectin (solid bars) was coated and BSA (column 1), rPKM1 (column 2), rPKM2 (column 3), or rPKM2+FBP (column 4) was added to the culture medium.

FIG. 4E is a bar graph showing cell spreading (relative to rPKM2) of HUVEC cells on microscopic chamber slide coated with ECM and in the presence of BSA (column 1), rPKM1 (column 2), or rPKM2 (column 3).

FIG. 5A is a bar graph showing tumor volume (mm³) of PC-3 tumor in mice treated with saline (-♦-), rPKM1 (-▪-), rPKM2 (-X-), or rPKM2+FBP (-▴-) as a function of time (days) after inoculation. FIG. 5B is a plot showing tumor weight (mg) of PC-3 tumor in mice treated with saline (column 1), rPKM1 (column 2), rPKM2 (column 3), or rPKM2+FBP (column 4) harvested after 13 days growth with 8 days treatment.

FIGS. 5C and 5D are bar graphs showing microvessel density (FIG. 5C) and the number of branch points (FIG. 5D) using antibody against CD31 on tissue sections prepared from PC-3 tumors treated with saline (column 1), rPKM1 (column 2), rPKM2 (column 3), or rPKM2+FBP (column 4).

FIG. 6A is a graph showing chromatography profiles (mAU at UV 280 nm as a function of elution volume (mL)) of a standard molecular weight calibration kit. FIG. 6B is a graph showing (mAU at UV 280 nm as a function of elution volume (mL)) of rPKM2 (solid line) and rPKM1 (dashed line) at concentration of 12 μM. FIG. 6C is a bar graph showing pyruvate kinase activity (relative to rPKM1) of rPKM1 (column 1), rPKM2 (column 2), and rPKM2+FBP (column 3) (5 μg/ml). FIGS. 6D-6F are chromatography profiles (mAU at UV 280 nm as a function of elution volume (mL)) of rPKM2 (FIG. 6D, at 1, 2, 4, 8 μM)), rPKM2+FBP (FIG. 6E, at 1, 2, 4, 8 μM), and rPKM1 (FIG. 6F, at 1, 2 μM).

The dimer and tetramer ratios (T/D ratio) in FIGS. 6D-6F were calculated by the areas under the dimer and tetramer peaks. FIG. 6G is a chromatography profile (mAU at UV 280 nm as a function of volume (mL)) of 1 μM rPKM2 (dashed line) or rPKM1 (solid line).

FIGS. 7A-7B are bar graphs showing proliferation (BrdU detection relative to saline) of SW620 (solid bars) or PC-3 cells (open bars) in the presence of saline (FIGS. 7A-7B, column 1), PabPKM2 (FIG. 7A, column 2), PabCon (FIG. 7A, column 3), 5 μg/ml rPKM1 (FIG. 7B, column 2), 5 μg/ml rPKM2 (FIG. 7B, column 3), or 5 μg/ml rPKM2+FBP (FIG. 7B, column 4).

FIG. 8A is a bar graph showing cell migration (relative to saline) of SW620 (solid bars) or PC-3 (open bars) cells in the presence of saline buffer (column 1), rPKM1 (column 2), rPKM2 (column 3), rPKM2+FBP (column 4) analyzed by Boyden chamber assay.

FIG. 8B is a bar graph showing cell attachment (relative to 620CM) of HUVEC cells to cell culture plate on which ECM was coated cultured in SW620 cell culture medium (620CM, column 1), medium without SW620 cell culturing containing PabPKM2 (ConCM+PabPKM2, column 2), SW620 cell culture medium containing PabPKM2 (620CM+PabPKM2, column 3), or SW620 cell culture medium containing IgG from preimmune serum (620CM+PabCon). FIG. 8C is a bar graph showing cell attachment (relative to saline) of SW620 cells to cell culture plate on which ECM was coated cultured in medium containing saline (column 2), rPKM1 (column 2), rPKM2 (column 3), or rPKM2+FBP (column 4).

### DETAILED DESCRIPTION

### I. Definitions

The term “angiogenesis” refers to the growth of new blood vessels from pre-existing vessels.

The term “soluble PKM2” refers to pyruvate kinase isoform M2 (PKM2) present in the circulation of a subject. The term does not include PKM2 that is present within intact cells.

The term “neutralize” refers to the ability of an agent, such as an antibody, to specifically bind a ligand and in so doing block or inhibit the ligand's biological activity. A neutralizing antibody is an antibody that inhibits or abolishes some biological activity of its target antigen.

The term “antibody” refers to natural or synthetic antibodies that binds or selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

A “monoclonal antibody” can be obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The term “specifically binds” refers to a binding reaction which is determinative of the presence of the antigen or receptor in a heterogeneous population of proteins and other biologics. Generally, a first molecule (e.g., antibody) that “specifically binds” a second molecule (e.g., antigen) has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “individual,” “host,” “subject,” and “patient” are used interchangeably to refer to any individual who is the target of administration or treatment. The subject can be a vertebrate, for example, a mammal. Thus, the subject can be a human or veterinary patient.

The term “therapeutically effective” refers an amount of composition that is sufficient to ameliorate one or more causes or symptoms of a disease or disorder. Such amelioration only requires a reduction or alteration, not necessarily elimination of the disease or disorder.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “neoplastic cells” refers to a cell undergoing abnormal cell proliferation (“neoplasia”). The growth of neoplastic cells exceeds and is not coordinated with that of the normal tissues around it. The growth typically persists in the same excessive manner even after cessation of the stimuli, and typically causes formation of a tumor.

The term “tumor” or “neoplasm” refers to an abnormal mass of tissue containing neoplastic cells. Neoplasms and tumors may be benign, premalignant, or malignant.

The term “metastasis” refers to the spread of malignant tumor cells from one organ or part to another non-adjacent organ or part. Cancer cells can “break away” from a primary tumor, enter lymphatic and blood vessels, circulate through the bloodstream, and settle down to grow within normal tissues elsewhere in the body.

### II. Compositions

A. Soluble PKM2 Binding Molecules

Soluble PKM2 binding molecules are disclosed for used in the disclosed compositions and methods. In certain embodiments, the PKM2 binding molecules specifically bind and neutralize circulating PKM2 in the subject.

1. Antibodies

In one specific embodiment, the PKM2 binding molecule is an antibody. Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability may not be evenly distributed through the variable domains of antibodies and may be concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the disclosed antibodies contain at least the CDRs necessary to bind and neutralize PKM2.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific for PKM2. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. Libraries of antibodies or active antibody fragments can also be generated and screened using phage display techniques.

Human and Humanized Antibodies

Many non-human antibodies (e.g., those derived from mice, rats, or rabbits) are naturally antigenic in humans, and thus can give rise to undesirable immune responses when administered to humans.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

Antibodies can be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

The antibody can be bound to a substrate or labeled with a detectable moiety or both bound and labeled. The detectable moieties contemplated with the present compositions include fluorescent, enzymatic and radioactive markers.

Single-Chain Antibodies

A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

Monovalent Antibodies

In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them.

Hybrid Antibodies

The PKM2 binding molecule may be a hybrid antibody. In hybrid antibodies, one heavy and light chain pair is homologous to that found in an antibody raised against one epitope, while the other heavy and light chain pair is homologous to a pair found in an antibody raised against another epitope. This results in the property of multi-functional valency, i.e., ability to bind at least two different epitopes simultaneously. Such hybrids can be formed by fusion of hybridomas producing the respective component antibodies, or by recombinant techniques. Such hybrids may, of course, also be formed using chimeric chains.

Conjugates or Fusions of Antibody Fragments

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

Method of Making Antibodies Using Protein Chemistry

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment.

3. Peptides

In some embodiments, the PKM2 binding molecule is a peptide. Peptides that specifically bind PKM2 can be identified using routine methods, such as phage display and yeast two-hybrid assays.

The disclosed peptides generally contain at least one segment that selectively binds PKM2. Such segments can be referred to as “PKM2-binding segments.” The disclosed PKM2-binding peptides can have a variety of lengths and structures as described herein. Generally, the lengths can range from peptide to polypeptide length, and all such lengths are encompassed as described herein. Merely for the sake of convenience, the disclosed peptides and polypeptides generally are referred to herein as “polypeptides” but it is intended that use of this term encompasses such compositions that could be considered peptides, unless the context clearly indicates otherwise.

In some cases, each PKM2-binding segment independently is about 4 to about 50 amino acids in length, including about 4, 5, 6, 7, 8, 9, 10, 11, 12, 13, 14, 15, 16, 17, 18, 19, 20, 25, 30, 35, 40, 45, or 50 amino acids in length. The PKM2-binding segment can have less than about 100 amino acid residues, including less than about 100, 95, 90, 85, 80, 75, 70, 65, 60, 55, 50, 45, 40, 35, 30, 25, 20 amino acid residues. The PKM2-binding segment can have more than about 8 amino acid residues, including more than about 8, 9, 10, 11, 12, 13, 14, 15, 16, 17, 18, 19, 20, 25, 30, 35, 40, 45, or 50 amino acid residues.

In order to increase efficiency, the disclosed PKM2-binding polypeptide can be polymeric. For example, Multiple Antigen Peptide System (MAPS), first described by Dr. James Tam as a method of presenting epitopes to the immune system, is based on a small immunologically inert core molecule of radially branching lysine dendrites onto which a number of peptide antigens are anchored. The result is a large macromolecule which has a high molar ratio of peptide antigen to core molecule and does not require further conjugation to a carrier protein.

Thus, the isolated polypeptide can have two or more PKM2-binding segments, including 2, 3, 4, 5, 6, 7, 8, 9, 10, 11, 12, 13, 14, 15 or more segments. In some aspects, the isolated polypeptide can have eight PKM2-binding segments. In some aspects, the isolated polypeptide is unbranched, wherein two or more segments are on the same linear polypeptide. In other aspects, the isolated polypeptide has two or more amino acid branches, including 2, 3, 4, 5, 6, 7, 8, 9, 10, 11, 12, 13, 14, 15 or more amino acid branches. Thus, the isolated polypeptide can have a peptidyl core of 2, 3, 4, 5, 6, 7, 8, 9, 10 or more branched lysine residues, wherein two or more of the PKM2-binding segments are linked to two or more branched lysine residues. In addition, each of the branches can be monomeric or polymeric.

The disclosed polypeptides can be artificial sequences and can be synthesized in vitro and/or recombinantly. The disclosed polypeptides can be peptides that are not naturally occurring proteins and can be peptides that have at least two contiguous sequences that are not contiguous in a naturally occurring protein. The disclosed polypeptides can be 5 to about 50 amino acids in length. The disclosed polypeptides can be less than about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 amino acids in length.

3. Combination Therapies

Numerous anti-cancer (antineoplastic) drugs are available for combination with the present method and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Flurocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-I a, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

4. Pharmaceutical Formulations

A pharmaceutical compositions containing therapeutically effective amounts of one or more of the disclosed PKM2 binding molecules, such as a PKM2 neutralizing antibody, in a pharmaceutically acceptable carrier is disclosed. Pharmaceutical carriers suitable for administration of the disclosed PKM2 include any such carriers known to those skilled in the art to be suitable for the particular mode of administration.

In addition, the compounds may be formulated as the sole pharmaceutically active ingredient in the composition or may be combined with other active ingredients. For example, the compounds may be formulated or combined with known anti-neoplastic drugs, NSAIDs, anti-inflammatory compounds, steroids, and/or antibiotics.

The PKM2 binding molecules may be formulated into suitable pharmaceutical preparations such as solutions, suspensions, tablets, dispersible tablets, pills, capsules, powders, or sustained release formulations.

In one specific embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

The dosage and schedule for administration of a therapeutic antibody used in the disclosed methods can be determined by one of skill in the art. For example, the dosage of the antibody can range from about 0.1 mg/kg to about 50 mg/kg, typically from about 1 mg/kg to about 25 mg/kg. In particular embodiments, the 4 antibody dosage is 1 mg/kg, 3 mg/kg, 5 mg/kg, 10 mg/kg, 15 mg/kg, 20 mg/kg or 25 mg/kg. The dosage schedule for administration of the antibody can vary depending on the desired aggressiveness of the therapy, as determined by the practitioner.

An exemplary treatment regime entails administration once per every two weeks or once a month or once every 3 to 6 months. Antibody is usually administered on multiple occasions. Intervals between single dosages can be weekly, monthly or yearly. Intervals can also be irregular as indicated by measuring blood levels of antibody to PKM2 in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

### III. Methods

Methods are disclosed for inhibiting angiogenesis in a subject that involve administering to the subject a composition containing an effective amount of soluble PKM2 binding molecules in a pharmaceutically acceptable excipent. In specific embodiments, the PKM2 binding molecules specifically bind and neutralize circulating PKM2 in the subject. In some of these embodiments, the composition contains an effective amount of PKM2 binding molecules to neutralize circulating PKM2 in the subject by at least 20% to 100%, including by about 20%, 30%, 40%, 50%, 60%, 70%, 80%, 90%, or 100%.

In some embodiments, the subject has detectable levels of PKM2 in a bodily fluid or stool when the PKM2 binding molecules are administered. It is expected that, if PKM2 has been detected in a bodily fluid or stool prior to treatment, e.g., less than 1 month before treatment, it will also be present in the subject at the time that treatment is initiated.

A. Inhibiting Angiogenesis

Angiogenesis inhibitors may be used therapeutically to combat diseases characterized by abnormal vasculature. It is a component of many diseases including cancer, blindness, and chronic inflammation.

Since tumors cannot grow beyond a certain size, generally 1-2 mm³, due to a lack of oxygen and other essential nutrients, they induce blood angiogenesis by secreting various angiogenic growth factors (e.g. VEGF). Angiogenesis is a necessary and required step for transition from a small harmless cluster of cells to a large tumor. Angiogenesis is also required for the spread of a tumor, or metastasis. Single cancer cells can break away from an established solid tumor, enter the blood vessel, and be carried to a distant site, where they can implant and begin the growth of a secondary tumor.

Angiogenesis in the eye underlies the major causes of blindness in both developed and developing nations. Angiogenesis occurs with exudative age-related macular degeneration (AMD), proliferative diabetic retinopathy (PDR), diabetic macular edema (DME), neovascular glaucoma, corneal neovascularization (trachoma), and pterygium. Neovascular or exudative AMD, the “wet” form of advanced AMD, causes vision loss due to abnormal blood vessel growth (choroidal neovascularization) in the choriocapillaris, through Bruch's membrane, ultimately leading to blood and protein leakage below the macula. Bleeding, leaking, and scarring from these blood vessels eventually cause irreversible damage to the photoreceptors and rapid vision loss if left untreated. Until recently, no effective treatments were known for wet macular degeneration. However, anti-angiogenic agents can cause regression of the abnormal blood vessels and improvement of vision when injected directly into the vitreous humor of the eye.

While diabetes management has largely focused on control of hyperglycemia, the presence of abnormalities of angiogenesis also cause or contribute to many of the clinical manifestations of diabetes. When compared with non-diabetic subjects, diabetics demonstrate vascular abnormalities of the retina, kidneys, and fetus. Diabetics have impaired wound healing, increased risk of rejection of transplanted organs, and impaired formation of coronary collaterals. In each of these conditions, and possibly in diabetic neuropathy as well, abnormalities of angiogenesis are implicated in the pathogenesis. A perplexing feature of the aberrant angiogenesis is that excessive and insufficient angiogenesis can occur in different organs in the same individual.

Angiogenesis is a common finding in chronic inflammatory diseases, such as asthma, rheumatoid arthritis, and psoriasis. Angiogenesis is a prominent feature of several CNS diseases including epilepsy and stroke. Evidence is also accumulating that angiogenesis is involved in the pathophysiology of multiple sclerosis and experimental autoimmune encephalomyelitis.

B. Treating Cancer

Thus, provided herein is a method of treating cancer in a subject, comprising administering to the cancer a composition that binds and neutralizes PKM2 in the circulation of a subject. The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. In some embodiments, the cancer is colorectal cancer.

C. Administration

The disclosed pharmaceutical compositions containing PKM2 binding molecules may be administered in a number of ways to achieve therapeutically effective amounts of the PKM2 binding molecules in the circulation of the subject. For example, the disclosed compositions can be administered intravenously, intraperitoneally, intramuscularly, subcutaneously, intracavity, or transdermally. The compositions may be administered parenterally, ophthalmically, vaginally, rectally, intranasally, or by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The disclosed compositions may be administered prophylactically to patients or subjects who are at risk for angiogenesis. Thus, the method can further comprise identifying a subject at risk for angiogenesis, e.g., tumor angiogenesis, prior to administration of the disclosed compositions.

The exact amount of the compositions required will vary from subject to subject, depending on the species, age, weight and general condition of the subject, the severity of the allergic disorder being treated, the particular composition used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the antibody used alone might range from about 1 μg/kg to up to 100 mg/kg of body weight or more per day, depending on the factors mentioned above.

The frequency of dose will depend on the half-life of the antibody molecule and the duration of its effect. If the antibody molecule has a short half-life (e.g. 2 to 10 hours) it may be necessary to give one or more doses per day. Alternatively, if the antibody molecule has a long half life (e.g. 2 to 15 days) it may only be necessary to give a dosage once per day, once per week or even once every 1 or 2 months.

### EXAMPLES

### Example 1

### Circulative PKM2 in Tumor Progression

Materials and Methods

Reagents, Cell Lines, Antibodies, and Protein Expression/Purifications

Antibodies against β-actin, mouse CD31, Ki-67 were purchased from Cell Signaling, SantaCruz, and Abcam respectively. Antibody against PKM2 was raised using recombinant PKM2 expressed/purified from E. coli. as an antigene. IgGs were purified from the rabbit anti-serum over a protein G column. Cell lines SW620 and PC-3 were purchased from ATCC, and HUVEC cells were purchased from Invitrogen. The cells were cultured by following the vendor's instructions. The cDNAs that encode human PKM2 and PKM1 were purchased from Adgenes. The cDNAs were subcloned into bacterial expression vector pEG-32a. The recombinant proteins were purified from bacterial lysates by a two column procedure.

Nude Mice Xenograft and Treatments

All animal experiments were carried out in accordance with the guidelines of IACUC of Georgia State University. Nude mice (nu/nu, Harlan Laboratory) were subcutaneously injected with 5×10⁶ of SW620 or PC-3 cells. Tumor formation and volumes were assessed every 2 days. Tumor volumes were measured by two perpendicular diameters of the tumors with the formula 4p/3×(width/2)²×(length/2). The tumor bearing mice were subjected to the i.p. injections of appropriate agents once every other days for eight days. The treatments started five days post tumor inoculations. The tumors were collected and weighed at the end of the experiments. Tissue sections were prepared from harvested tumors, and stained using commercially available antibodies against Ki-67 or mouse CD31. Statistical analyses were done in comparison to the control group with a paired Student's t test.

Results

A molecular signature of tumor development is that a shift in expression of isoenzymes of pyruvate kinases occurs to the tumor of almost all types. After four week xenograft tumor growth in nude mice, blood samples from the tumor-bearing mice were collected. PKM2 levels in the blood samples were analyzed by immunoblot of the serum. It was evident that the PKM2 levels in blood of the SW620 tumor mice were very high. As a control, no PKM2 was detected in blood of mouse without tumor inoculation. We also examined the PKM2 levels in the cell culture medium of SW620 cells. In consistent, we observed high levels of PKM2 in the medium.

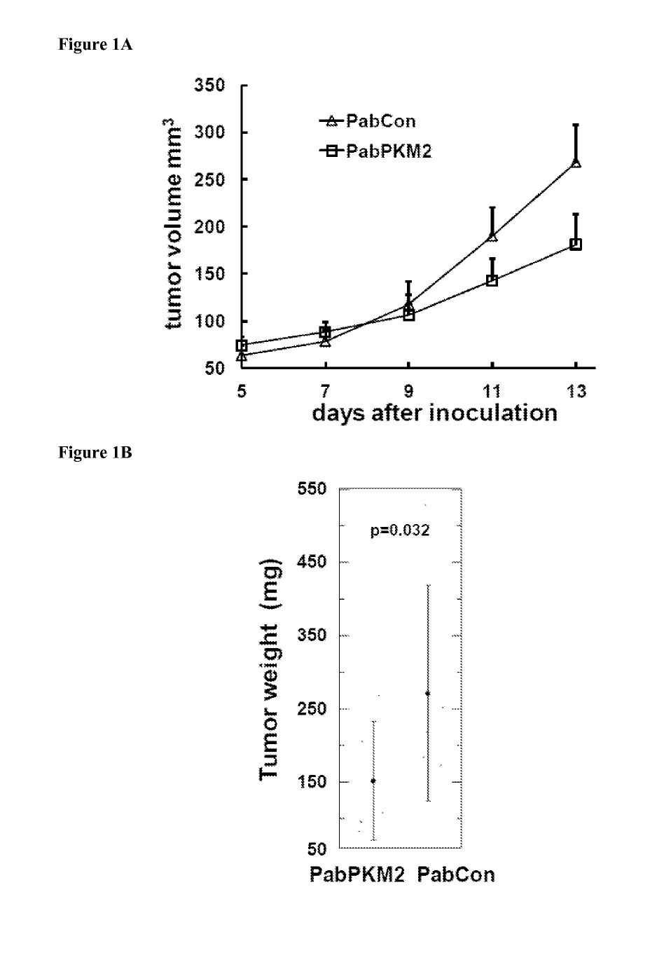

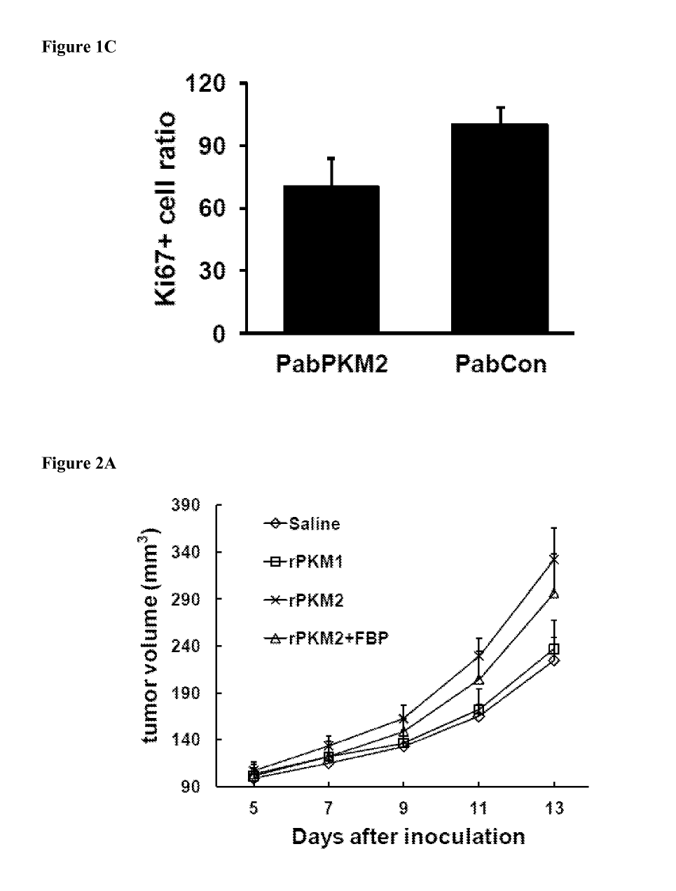

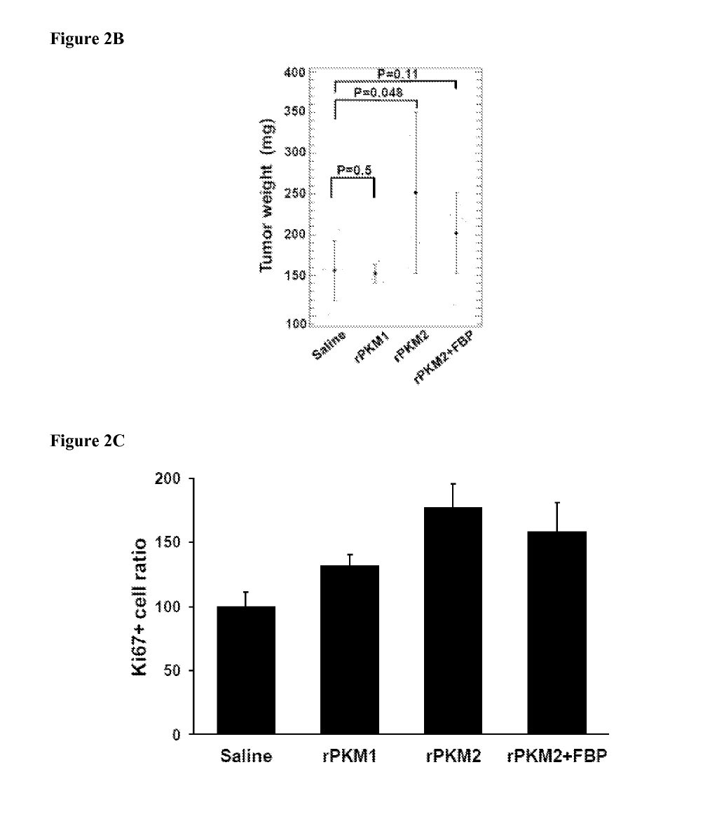

An in-house developed rabbit polyclonal antibody was raised against full length recombinant PKM2 (Ref to as PabPKM2). Antibody screening indicated specific recognition of PKM2 in the cell extracts. The recognition of cellular PKM2 by the antibody was completely abolished by the bacterially expressed PKM2. The antibody did not recognize any protein in serum collected from nude mouse. IgGs were purified from the antiserum of the PabPKM2 or rabbit pre-immune serum by a protein A/G bead column. The purified IgGs was i.p. injected into nude mice that carried xenograft tumor of SW620 cells every two days for 8 days. It was clear that the purified IgGs from the PabPKM2 greatly inhibited the tumor growth, while administration of the purified IgGs from the pre-immune serum did not exhibit any significant effects on the growth of the same xenograft tumor (FIGS. 1A, 1B). The results suggest that PKM2 in the blood circulation is critical important for the tumor growth.

### Example 2

### PKM2 Promotes Tumor Growth

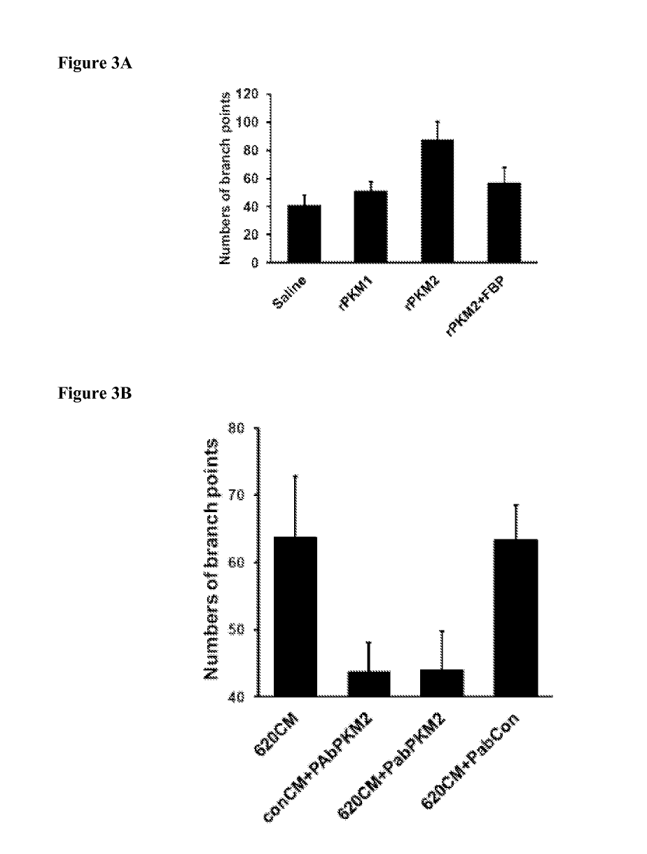

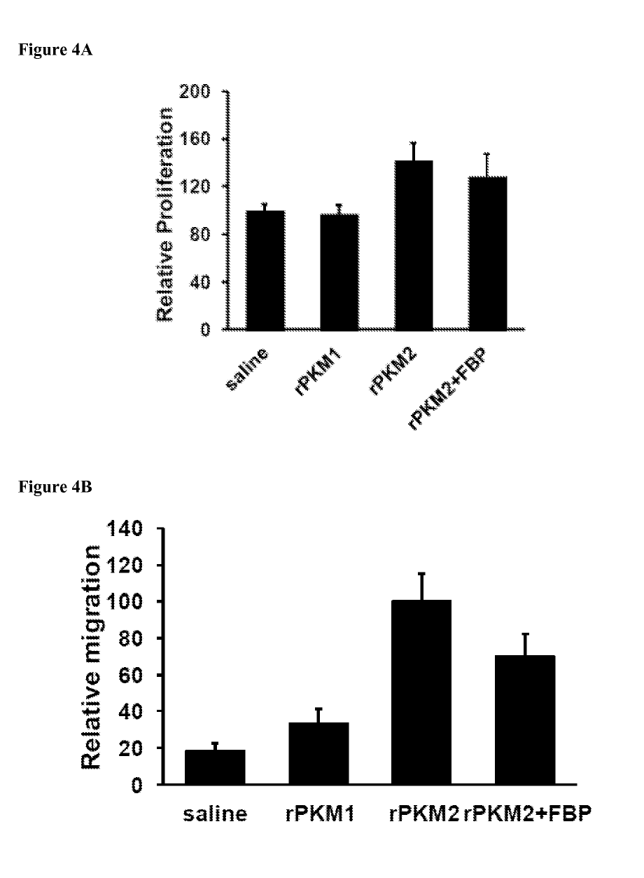

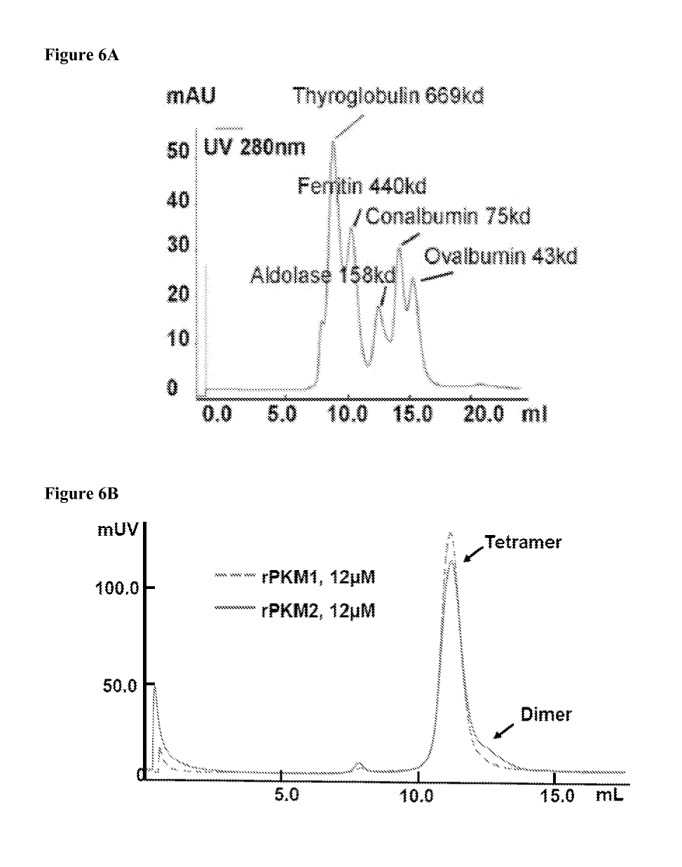

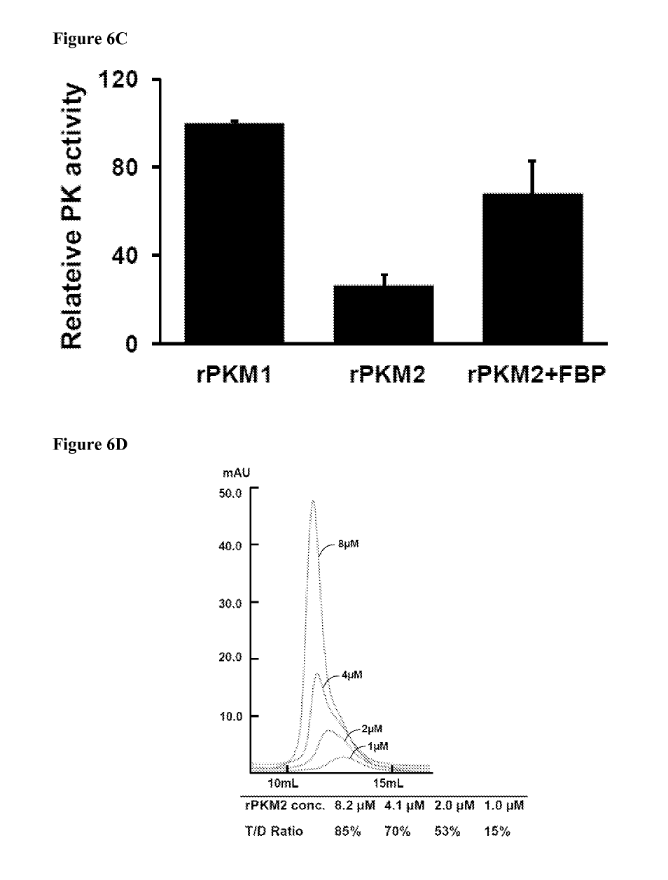

Bacterially expressed recombinant PKM2 (ref to as rPKM2) and its isoenzyme PKM1 (ref to as rPKM1) was used as a control. Since PKM2 is secreted from cancer cells, presumably, the protein should be present in the extra-cellular space of tumors. Thus, the purified rPKM2 and rPKM1 were pre-mixed with cancer cells at concentration of 2 μM. The mixtures were then s.c. implanted into nude mouse. The purified recombinant proteins were also subsequently i.p. injected (5 mg/kg) to the tumor-bearing nude mice every other days for 8 days. The first injection started 5 days post tumor inoculation. Clearly, the SW620 tumors that were treated with the rPKM2 experienced substantially higher growth rates compared to the tumors that were treated with the rPKM1 and buffer. The tumors treated with the rPKM1 and buffer saline had almost similar growth rates (FIGS. 2A, 2B). To test whether the observed effects of the rPKM2 was specific to the SW620 tumor only, we employed another xenograft model, human prostate cancer PC-3 cells, by the same treatment schedule. PKM2 was detected in the cell culture medium of PC-3 cells. It was clear that administration of the rPKM2 facilitated PC-3 tumor growth (FIGS. 5A, 5B).

### Example 3

### Effects of PKM2 Dimer and Tetramer Status in Promoting Tumor Growth

Materials and Methods

Size-Exclusion Chromatography

Size exclusion chromatograph was performed with a Superdex 200 10/300GL column. The samples of mouse serum (2-8 mg/ml of total protein), the rPKM2 (˜15 μM), the rPKM1 (˜15 μM) were prepared in tris-HCl buffer with/without FBP. 100 μl of the sample was loaded into the column and eluted with elution buffer (50 mM phosphate, 0.15M NaCl pH7.2). The fraction of 300 μl was collected, and 20 μl of each fraction was analyzed by immunoblot. The elution profiles were compared to that of a size exclusion chromatograph calibration kits (GE Healthcare) under identical conditions. The elution profile was plotted against Log MW according to vendor's instructions.

Pyruvate Kinase Activity

Pyruvate kinase activity was analyzed by following an experimental procedure previously described (Christofk H R, Nature, 452:181 (2008)).

Results

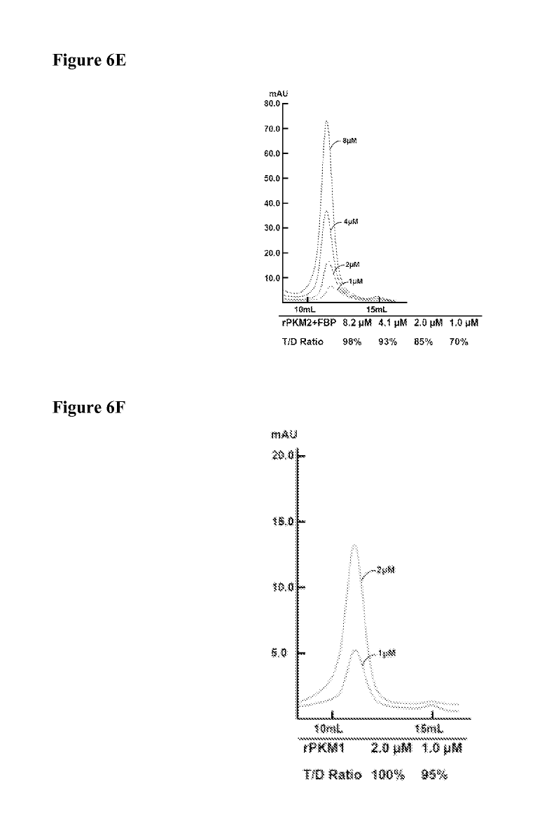

It is believed that the PKM2 in the cancer patient blood circulation exists as a dimer (Hugo F, et al. Anticancer Res 19:2753 (1999); Wechsel H W, et al. Anticancer Res 19:2583 (1999)), while the protein in cancer cells exists as a mixture of tetramer and dimer (Hitosugi T, et al., Sci Signal 2:ra73 (2009); Mazurek, S. Ernst Schering Found Symp Proc, 99 (2007)). Thus, an interesting issue is whether the dimer and tetramer status of PKM2 have different effects in promoting tumor growth. Chromatography analyses followed by immunoblots indicated that the rPKM2 existed mostly as dimer in the circulation, while the rPKM1 was mostly tetramer. Using an ELISA analysis, it was estimated that the concentration of the i.p. injected rPKM2 and rPKM1 (at dose of 5 mg/kg) in the mouse blood circulation was around 500-800 nM 4 hours after the administration. Chromatography profiles indicated that the rPKM2 existed as a mixture of tetramer and dimer (with tetramer to dimer ratio at around 80% to 20%) at concentration of 12 μM, while the rPKM1 was almost completely tetramer at the same concentration (FIG. 6B). The purified proteins possessed pyruvate kinase activity (FIG. 6C). Interestingly, dilution of the rPKM2 led to conversion of tetramer to dimer with the rPKM2 became almost completely dimer at around 1 μM (FIGS. 6D, 6G). This concentration is very close to the concentration of the administered rPKM2 in mouse blood circulation. Most of the rPKM1 still existed as tetramer at this concentration (FIGS. 6E, 6G). Addition of 3 mM FBP converted the rPKM2 to the tetramer, even at concentration as low as 1 μM (FIG. 6F). Consistently, a large portion of rPKM2 was tetramer in mouse blood circulation when the protein was co-administered with 3 mM FBP. Thus, we questioned whether addition of FBP would affect the effects of PKM2 on facilitating tumor growth. The addition of FBP to the rPKM2 reduced the effects of rPKM2 on promoting tumor growth both with the SW620 and PC-3 tumors (FIGS. 2A, 2B, 5A, 5B). This was consistent with the fact that FBP facilitates the PKM2 dimer to tetramer conversion.

### Example 4

### PKM2 Promotes Angiogenesis

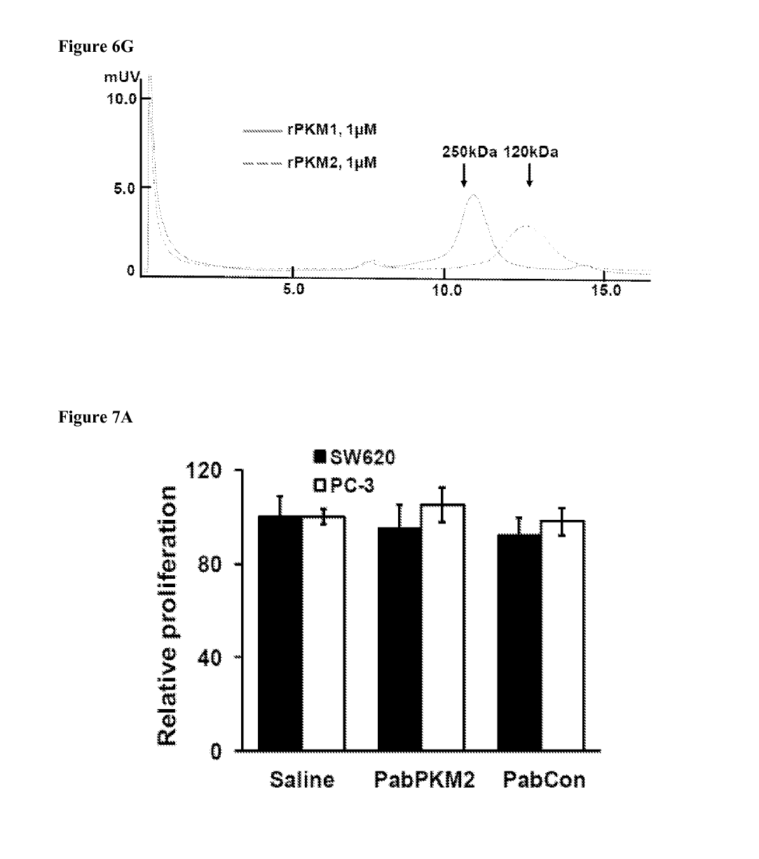

It is intriguing that cancer cells release PKM2 to the blood circulation and the circulative PKM2 promotes cancer growth. We questioned what the functional role of the circulative PKM2 is in promoting tumor growth. One possibility is that extra-cellular PKM2 promotes cancer cell proliferation. Tissue section stains with an antibody against Ki-67 indeed indicated that the tumors treated with the PabPKM2 had reduced proliferation rates, while the tumors treated with rPKM2 had higher proliferation rates (FIG. 2C). However, addition of the purified IgGs from the PabPKM2 and the rabbit pre-immune serum into SW620 and PC-3 cell culture medium did not lead to cell proliferation. Similarly, the proliferation of SW620 and PC-3 cells did not experience any significant change upon treatments with the rPKM2, rPKM1, and rPKM2+FBP (FIGS. 7A, 7B). Thus, the tumor growth promotion by the rPKM2 and inhibition by the PabPKM2 were unlikely due to their actions on cancer cells.

The other possibility is that tumor cells release PKM2, and the PKM2 in the blood circulation feedback promotes angiogenesis to facilitate tumor growth. To test this conjecture, we carried out histology analyses with the tumor tissue sections using antibody against mouse CD31, a marker for endothelial cells. It was very clear that treatment of mouse with the IgGs purified from the PabPKM2 dramatically reduced blood vessels in the xenograft of SW620 (Table 1). Reversely, treatment of tumor bearing mouse with the rPKM2 led to substantial increases in blood vessels in both PC-3 and SW620 tumors. The rPKM2+FBP had reduced effects comparing to those of the rPKM2 treated group, while the rPKM1 had no significant effects (Table 2, FIGS. 5C, 5F).

Table 1 shows quantitative analyses of vessel lengths, densities, and branch points (manually counting) of the CD31 staining of the tumor tissue sections using software imaging-J. The quantization was statistical mean values of randomly selected 4 fields in randomly selected 3 sections from each tumor.

TABLE 1
Quantitative analyses of tumor treated with PabPKM2 or PabCon

| | PabCon | PabPKM2 |
|---|---|---|
| MVD (per mm²) | 61.5 ± 9.8 | 37.2 ± 7.1 |
| Vessel Length (μm) | 435.6 ± 30.9 | 328.5 ± 44.1 |
| Branch Points | 4.3 ± 2.8 | 3.5 ± 2.0 |

Table 2 shows quantitative analyses of vessel lengths, densities, and branch points (manually counting) of the CD31 staining of the tumor tissue sections using the software imaging-J. The quantization was statistical mean values of randomly selected 4 fields in randomly selected 3 sections from each tumor.

TABLE 2
Quantitative analyses of tumor treated with recombinant proteins

| | Saline | pPKM1 | rPKM2 | rPKM2 + FBP |
|---|---|---|---|---|
| MVD | 41.4 ± 7.1 | 46.2 ± 11.9 | 90.6 ± 19.5 | 56.8 ± 16.5 |
| (per mm²) | | | | |
| Vessel | 352.6 ± 45.8 | 478.5 ± 39.6 | 933.3 ± 66.2 | 742.2 ± 92.8 |
| Length (μm) | | | | |
| Branch | 5.6 ± 2.2 | 4.3 ± 3.1 | 9.4 ± 4.5 | 8.0 ± 3.5 |
| Points | | | | |

### Example 5

### PKM2 Promotes Endothelial Cell Tube Formation

Materials and Methods

Endothelial Tube Formation Assays

Endothelial tube formations were carried out with the endothelial tube kit (Invitrogen). Briefly, HUVEC cells were seed in culture plat coated with martigel. After 30 minutes incubations, agents, e.g. FBS, proteins, or cancer cell culture medium, were added to the HUVEC. The cells were further cultured for additional 16 hours. The formed endothelial tubes were analyzed under light microscope. For the tube formation with supplement of SW620 culture medium, no FBS was added to the HUVEC cell culture.

Results

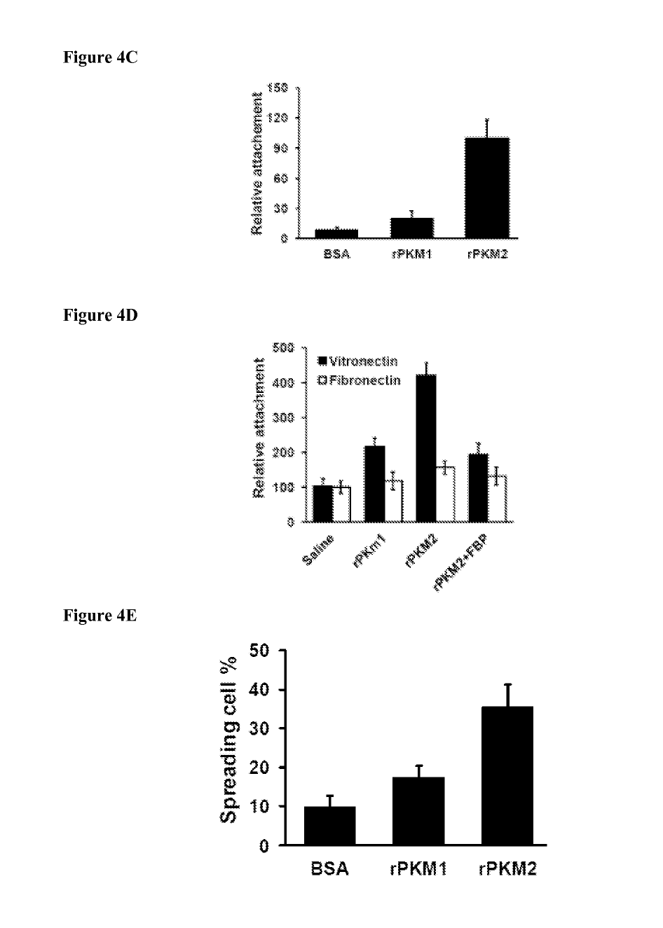

To further verify the role of PKM2 in promoting angiogenesis, we employed the in vitro tube formation assay using HUVEC cells. The rPKM2, the rPKM2+FBP, the rPKM1, and buffer alone were added to the culture medium of the cells. Formation of the endothelial tubes was analyzed. It was clear that the rPKM2 strongly promoted endothelial tube formation both in tube density and the sprouts of the formed tubes (FIG. 3A). The time required for formation of the tubes was also substantially shortened, and the formed tubes were maintained much longer time. The rPKM2+FBP had less effects compared to that of the rPKM2. The rPKM1 had only marginal effects, while buffer saline had no effects (FIG. 3A). We subsequently tested the effects of the PabPKM2 on the tube formation by co-culture the IgG from the anti-serum and the medium collected from SW620 cell cultures with HUVEC cells. Immunoblots indicated that PKM2 in SW620 cell culture medium was removed by the addition of the antibody PabPKM2. Clearly, the antibody greatly reduced the endothelial tube formation in the co-culture of SW620 medium with HUVEC cells (FIG. 3B). These in vitro tests supported our notion that PKM2 promotes angiogenesis.

### Example 6

### PKM2 Promotes Angiogenesis by Facilitating Endothelial Cell Migration and Cell Adhesions to Extracellular Matrix

Materials and Methods

Boyden Chamber and Cell Proliferation Assays

QCM™ 24-Well Fluorimetric Cell Migration Assay kit (ECM) was used to measure the migration of different cells. The test cells were first treated under the different conditions (indicated in figure legends) in regular cell culture plates. The treated cells were re-suspended into optimum medium (without serum) and seeded into the inner chamber of the migration assay kit. The culture medium with 10% FBS was added to the outer chambers. After overnight incubation, medium in the inner chamber was removed and the cells attached to the outer bottom side were detached using the cell detachment buffer (included in the kit). The detached cells were then lysed using the cell lysis buffer (included in the kit). The amounts of the migrated cells were determined by measuring the fluorescence using λex=485 nm and λem=535 nm. For analyses of cell proliferation, a cell proliferation ELISA kit that measures BrdU incorporation was used. Briefly, cells were incubated for appropriate time in the presence of 10 μM BrdU under different conditions (indicated in figures). The cells were fixed after incubation and washed 3 times. The fixed cells were detected by anti-BrdU-POD antibody and secondary antibody. The nuclei incorporations of BrdU were measured by chemiluminescence emission (Victor 3™, PerkinElmer). Cell proliferation was also measured by cell number counting. Cells were incubated for appropriate time under appropriate conditions. Cell numbers was counted before and after the indicated time of culture by three independent cell counting.

Attachment Assays

The cells were cultured overnight under standard conditions. Next days, different cells (with appropriate cell numbers) were transferred to a new plate with wells that coated with different proteins (indicated in the figures) with fresh medium with addition of appropriate agents in the medium (indicated in the figures). The cells were further cultured for 2 hours and washed gently. The attached cells were either directly counted or lysed. The cell lysates were then measured to determine the amounts of attached cells.

Cell Spreading Assay

Cells were seeded into a 24-well plate coated with ECM at a density of 1×10₅ cells per well and fixed after 3 hours. Spread and non-spread cells were counted in five representative fields. Nonspread cells were defined as small, round cells with little or no membrane protrusions, whereas spread cells were defined as large cells with obvious membrane protrusions and visible lamellipodia.

Results

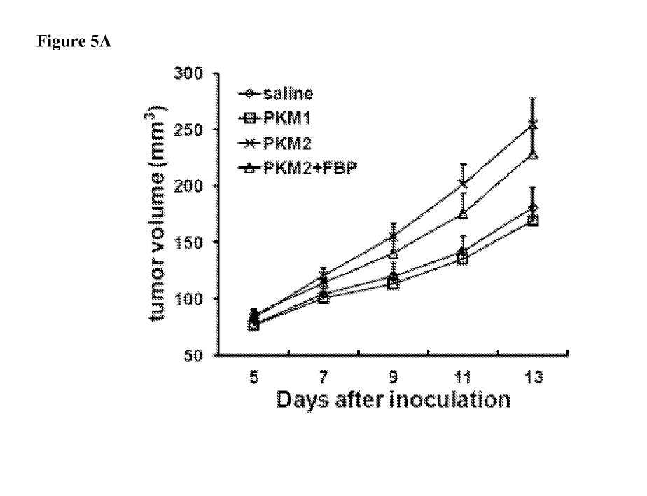

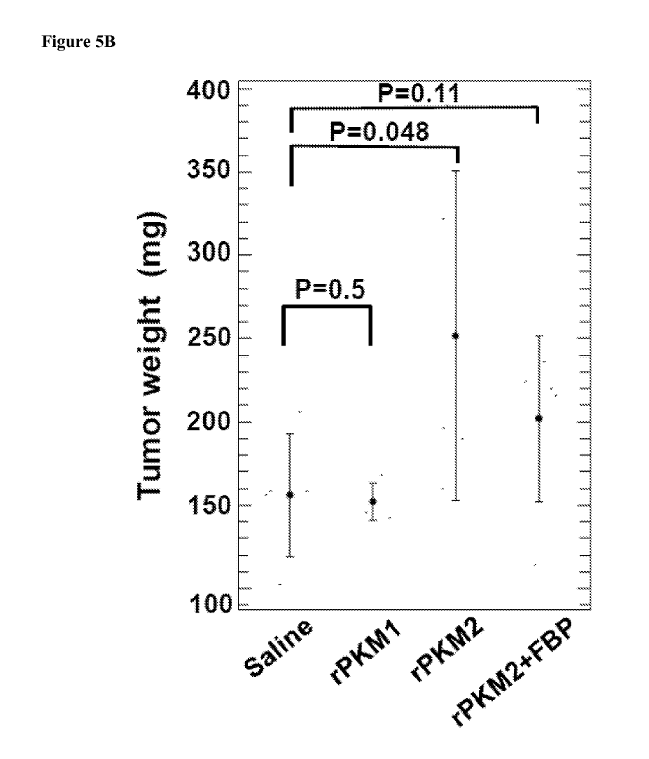

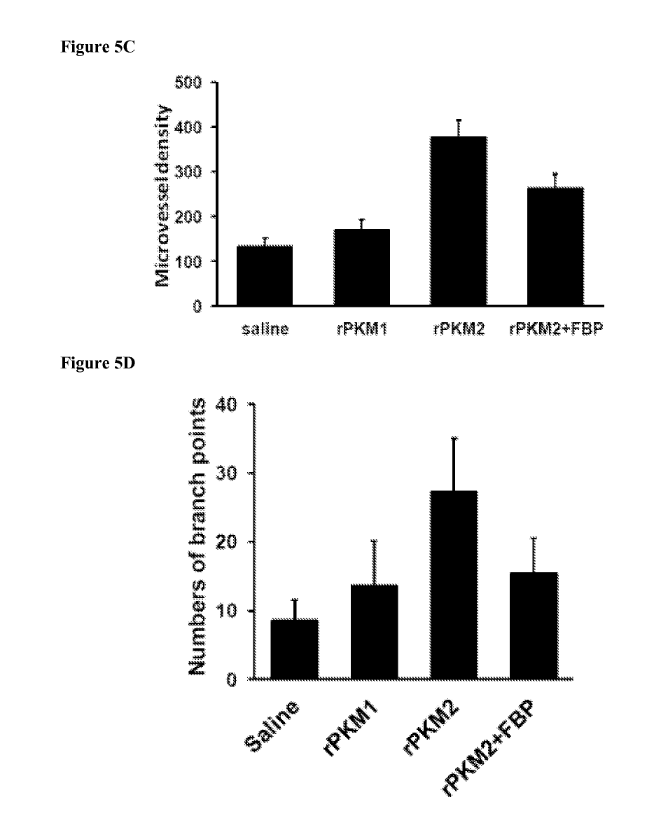

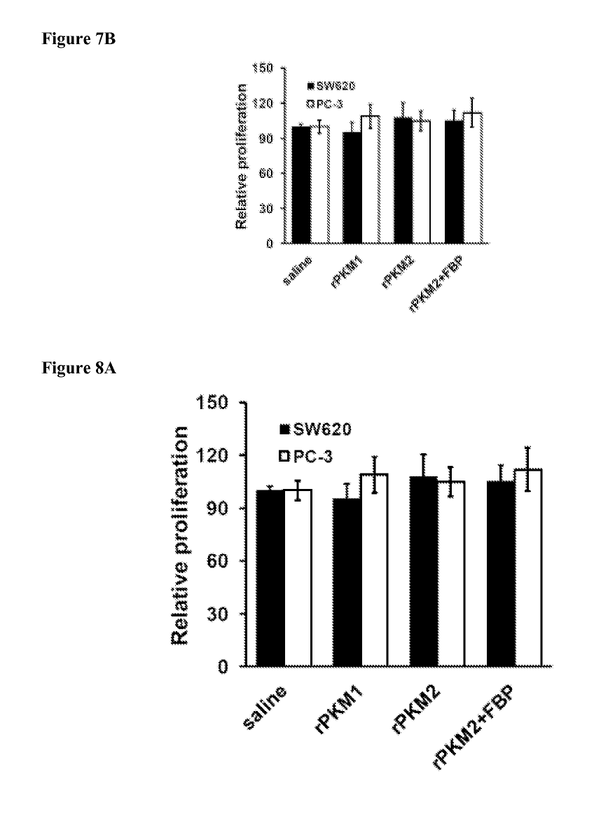

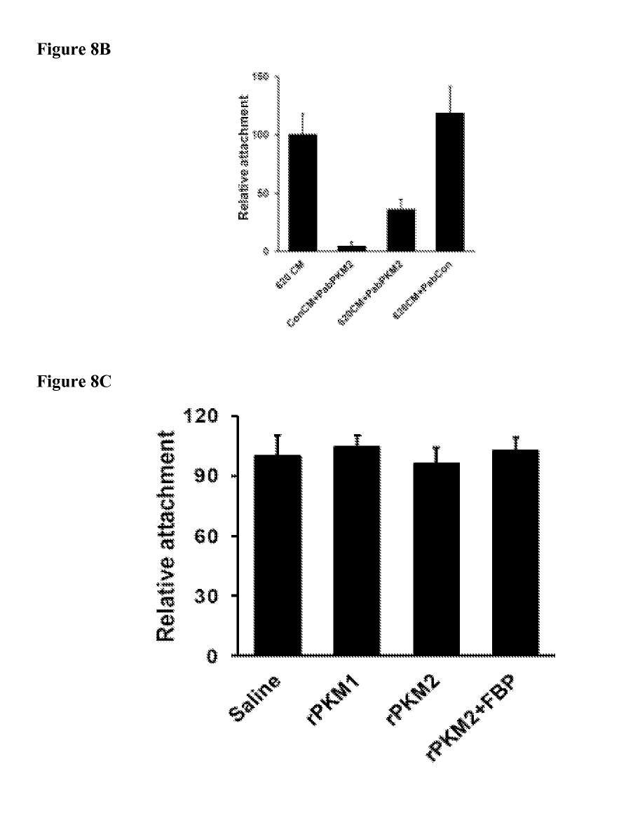

Addition of rPKM2, rPKM2+FBP, and the rPKM1 to the cell culture medium led to a marginal increase in cell proliferation (FIG. 4A). It is unlikely this small effect would be the sole factor that confers the dramatically in vivo effects on tumor growth. On the other hand, boyden chamber assays showed that addition of the rPKM2 led to a strong increase in cell migration, and the effects were substantially reduced with addition of rPKM2+FBP. FBP alone and the rPKM1 had almost no effects (FIG. 4B). The increases in cell migration were not observed with both SW620 and PC-3 cells (FIG. 8A). There was a significant change in the attachment of HUVEC cells to the culture plates with the rPKM2 coated to the plate (FIG. 4C). In addition, the attachment of HUVEC cells to ECM coated plate was substantially strengthened by addition of SW620 cell culture medium, while the enhancement was abolished by addition of the antibody PabPKM2 (FIG. 8B). It was clear that the cell adhesion to vitronectin, a matrix molecule known to be essential for the integrin αvβ3 mediated endothelial cell adhesion, spreading, and migration on the extracellular matrix (12), was strongly enhanced upon the addition of the rPKM2 to the culture medium (FIG. 4D). The effects were not observed with addition of the rPKM1. A strong effect was observed on the HUVEC cell spreading by addition of rPKM2 into the cell culture medium, while this effect was not observed with rPKM1 (FIG. 4E). The effects of PKM2 on cell adhesion to vitronectin and cell migration was not observed with epithelial cancer cells (FIGS. 8A, 8C), indicating that the effects were endothelial cell specific.

A very high percentage of cancer patients of different cancer types have elevated levels of PKM2 in their blood circulation. The circulative PKM2 μlays a critical role in facilitating tumor growth by promoting tumor angiogenesis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed:

1. A method of inhibiting angiogenesis in a subject, comprising administering to the subject an effective amount of a composition comprising pyruvate kinase isoform M2 (PKM2) binding molecules, wherein the PKM2 binding molecules binds circulating PKM2 in the subject.

2. The method of claim 1, wherein the PKM2 binding molecules are administered to the subject and the subject has detectable levels of PKM2 in a fluid or stool.

3. The method of claim 1, wherein the PKM2 binding molecules reduce circulating PKM2 in the subject by at least 20%.

4. The method of claim 1, wherein the PKM2 binding molecules having antibodies.

5. The method of claim 1, wherein the PKM2 binding molecules having proteins or peptides.

6. The method of claim 1, wherein the PKM2 binding molecule is a peptide.

7. The method of claim 1, wherein the subject has cancer, wherein the method inhibits angiogenesis in a solid cancer.

8. The method of claim 1, wherein the subject has exudative age-related macular degeneration (AMD), wherein angiogenesis in inhibited in the eye of the subject.

9. A pharmaceutical composition, comprising
(a) antibodies that bind pyruvate kinase isoform M2 (PKM2) in an effective amount to reduce soluble PKM2 of a human subject, and
(b) a pharmaceutically acceptable carrier.

10. The pharmaceutical composition of claim 9, wherein the antibodies comprise humanized antibodies.
